(19) United States
(12) Patent Application Publication
(10) Pub. No.: US 2014/0212494 A1
(43) Pub. Date: Jul. 31, 2014

(54) Methods of Improving Digestive Health

(71) Applicant: Purdue Research Foundation, West Lafayette, IN (US)

(72) Inventors: Bruce Hamaker, West Lafayette, IN (US); Ali Keshavarzian, Evanston, IL (US); Mark D. Cisneros, West Lafayette, IN (US); Amandeep Kaur, Frederick, MD (US); Heather Rasmussen, Chicago, IL (US)

(21) Appl. No.: 13/625,711

(22) Filed: Sep. 24, 2012

Related U.S. Application Data
(60) Provisional application No. 61/537,873, filed on Sep. 22, 2011.

(51) Int. Cl. A61K 31/718; A61K 9/16

(52) U.S. Cl. CPC A61K 31/718; A61K 9/16
USPC 424/489; 514/60

(57) ABSTRACT

A method of treating constipation is disclosed. The method includes administering starch-entrapped microbeads.

### PRIORITY

The present U.S. Non-provisional patent application is related to and claims the priority benefit of U.S. Provisional Patent Application Ser. No. 61/537,873, filed Sep. 22, 2011, the contents of which is hereby incorporated by reference in its entirety into this disclosure.

### STATEMENT REGARDING GOVERNMENT FUNDING

This invention was made with government support under DK088525 awarded by the National Institutes of Health. The government has certain rights in the invention.

### TECHNICAL FIELD

The present disclosure generally relates to methods of treating gastrointestinal disorders.

### BACKGROUND

Recent studies provide compelling evidence that inflammation is the underlying mechanism of many common disorders such as metabolic syndrome (MetS) (Delzenne N M, et al. Microb Cell Fact 2011; 10 Suppl 1:S10; Cani P D, et al. Diabetes 2007; 56:1761-72; Cani P D, et al. Diabetes 2008; 57:1470-81) and obesity, as well as diseases like CVD (Manco M, et al. Endocr Rev 2010), diabetes (Delzenne et al. Microb Cell Fact 2011; 10 Suppl 1:S10., Cani P D, et al. Diabetes 2008; 57:1470-81; Giongo A, et al. Isme J 2011; 5:82-91), Alzheimer's disease (Zhang R, et al. J Neuroimmunol 2009; 206:121-4), and cancer (Rose D J, et al. Nutr Rev 2007; 65:51-62, Fasano A. et al. Physiol Rev 2011; 91:151-75). The discovery that gut-derived endotoxin (LPS) is the most likely trigger for systemic inflammation has highlighted the critical role of gut microbiota because the microbiota are the source of this endotoxin which may leak in chronic diseases (Delzenne N M, et al., Microb Cell Fact 2011; 10 Suppl 1:S10.; Frazier T H, et al. J Parenter Enteral Nutr 2011; 35:14S-20S.) Attempts to modify microbiota in the gut to prevent endotoxin-initiated inflammation and metabolic disarray by dietary probiotic and prebiotic supplementation has received a great deal of attention (Frazier T H, et al. Br J Nutr 2010; 104 Suppl 2:S1-63; Macfarlane G T, et al. J Clin Gastroenterol 2011; 45 Suppl: S120-7).

In food science, probiotics are used to deliver living bacterial cells to the gut of humans (and other animals) to adjust the gut ecosystem to improve health or provide health benefits. The use of probiotics is based on the concept that there is a healthy balance of bacteria in the intestinal tracts and that dysbiosis, the disruption of that balance, can result in illness. Probiotics, for example, are used to restore the balance of gut flora which may be disrupted by antibiotic treatment. Probiotics commonly include strains of Lactobacillis and Bifidobacterium which are considered to be beneficial to digestive health. Prebiotics, on the other hand, are non-digestible foods which promote the growth of such beneficial bacteria in the gut. Prebiotics include various oligosaccharides, including fructooligosaccharides, inulins, lactilol, lactosucrose, lactulose, and polydextrins which in some way stimulate the growth of beneficial gut bacteria. Prebiotics are commonly used in the U.S. with 2012 revenue estimated at $146.7 million (Frost S. Market Research 2011). However, despite common usage, prebiotics can still be viewed as nascent with limited knowledge of mechanism of action. This mechanistic knowledge gap has led to “lumping” all prebiotics into one group, resulting in sub-optimal outcomes for this potentially effective intervention.

Moreover, the rationale for use of a particular prebiotic is largely based on data derived from animal studies examining changes in the number of stool bifidobacteria (Gibson G R, et al. Nutr Res Rev 2004; 17:259-75.; Cani P D, et al. Diabetologia 2007; 50:2374-83; Martinez I, et al. Appl Environ Microbiol 2009; 75:4175-84). However, prebiotics (a term used here to encompass the broader concept of fermentable fiber and its beneficial functions), may have a much broader impact on inflammation and overall health beyond their effects on bifidobacteria and lactobacillus populations which comprise on the order of only 2-3% of the total gut microbiome. (Delzenne N M, et al.; Microb Cell Fact 2011; 10 Suppl 1:S10.; Roberfroid M, et. al. Br J Nutr 2010; 104 Suppl 2:S1-63.) Indeed, the intestinal microbiome is very diverse, composed of over 400 phylotypes (Frank D N, et al. Proc Natl Acad Sci USA 2007; 104:13780-5); thus, focusing on a limited subset of microbiome changes (i.e., bifodobacteria and lactobacillus) may be masking important and highly relevant changes in the gut. While recognized prebiotics such as fructooligosaccharides (FOS), inulin, and galactooligosaccharides (GOS) are known to promote the growth of beneficial bacteria, particularly bifidobacteria and lactic acid bacteria, many prebiotics and oligosaccharides have specificity of promoting certain narrow or broader groups of colonic bacteria (Roberfroid M, et al.; Macfarlane G T, et al.) For instance, butyrogenic or propiogenic substrates may target the growth of bacteria that either directly produce these SCFAs or provide an intermediate substrate for these bacteria (Rose D J, et al.)

Prebiotic structure is important for function; thus, the selection of an appropriate prebiotic may determine efficacy. For example, the widely-used prebiotics FOS and inulin are rapidly fermented in the proximal colon (Louis P, et. al FEMS Microbiol Lett 2009; 294:1-8; Kootte R S, et. al. Diabetes Obes Metab 2012; 14:112-20) where in rodents the majority of microbiota are present and fermentation normally occurs (Macfarlane G T, et al.) Not surprisingly, these products effectively improve pathological endpoints in rodent models of disease (Roberfroid M, et al. Rose D J, et al.). In contrast, most microbiota are present in the distal colon of humans (Macfarlane G T, et. al. J Appl Bacteriol 1992; 72:57-64); thus, FOS and inulin are likely not optimal for use in humans. Instead, the fermentable substrate should be slow and delayed in order to reach the distal colon in order to achieve the most beneficial effects on host health. We have shown that by modifying fiber structure, substrate fermentation profiles can be altered to favor slow, yet complete and delayed-onset fermentation (Rose D J, et al.; J Agric Food Chem 2010; 58:493-9). Structural differences causing alterations in fermentation profiles may also confer different biological functions. For example, rapidly fermenting prebiotics deprive the distal colon of fermentable substrates and promote protein putrification and production of toxic and injurious substrates (e.g., indols, phenols, and thiols) (Kootte R S, et al.). Furthermore, prebiotic-enhanced production of short chain fatty acids (SCFA), especially propionate and butyrate, by the intestinal microbiota, is thought to be a major driving mechanisms for the beneficial health effects of prebiotics. In contrast, alternative catabolic pathways result in production of BCFA which are thought to be potentially harmful to human health (Rose D J et al.; Louis P, et al.). The rate and amount of SCFA production depends on the types and relative amounts of bacterial taxa present in the colon, the substrate source, and gut transit time (Wong J M, et. al J Clin Gastroenterol 2006; 40:235-43). Finally, slow fermenting fibers also are more tolerable, permitting higher in vivo dosing (Kaur A, et. al Journal of Food Science 2011; 76:H137-H142).

The importance of dietary fiber in human diet and its numerous physiological functions, as well as role in the prevention and treatment of certain diseases including cardiovascular, diabetes and obesity, and more importantly those related to colon health: inflammatory bowel disease (IBD), diverticulosis and colon cancer, is well recognized. Fiber that is consumed by individuals may include natural fiber from the foods eaten as well as fiber generated from other sources added to a given food (added fiber). Fiber has also been categorized as fermentable and non-fermentable based on the extent of its fermentation in the colon.

Health benefits are derived from both non-fermentable fiber (among others: increased fecal bulk-affecting fecal output, dilution and increased transit time of carcinogens and toxins in the colon, increased bile salt binding there by influencing cholesterol levels in the blood, and increased digesta viscosity) and fermentable fiber (production of short chain fatty acids that most importantly prevent the growth of harmful bacteria and decrease the inflammatory response). Fiber fermentation produces short chain fatty acids (e.g., acetic, propionic, and butyric acids) that contribute to colon health by increasing blood flow, improving mineral and water absorption by maintenance of low luminal pH. Butyrate has also been shown to have a positive influence on epithelial metabolistarch-entrapped microbeads, cell cycling, the immune response, and intestinal motility which includes aiding in the relief of symptoms of constipation. Two aspects of fermentable fiber that have received attention are preferable production of butyrate, which has promising effects for treatment of colon disorders including irritable bowel disease and colon cancers, and slow fermentation rate so that fermentation occurs in both the proximal part of the colon, and in the distal part of the colon (where most cancer lesions are known to occur). Most fermentable fibers are very rapidly fermented in the proximal part of the colon with very little fermentation in the distal part. This rapid fermentation is believed to cause numerous side effects like bloating which is found with many fibers of the prior art including psyllium. These side effects are the primary reason why humans do not consume sufficient fiber to maintain adequate gut health.

There is a need in the art for the development of dietary fibers that preferably generate butyrate on fermentation and that are available for fermentation throughout the length of the colon (both proximal and distal) and ferment at a rate which reduces or minimizes bloating. Starch-based dietary fiber offers a distinct advantage because their fermentation produces proportionally more butyrate than traditional dietary fibers. Starch-based dietary fiber can also function as a prebiotic to stimulate the growth of beneficial gut bacteria, including for example Lactobacillis and Bifidobacterum strains.

A starch-based fiber material which exhibits slow fermentation, such that fermentation occurs over the length of the colon, would be of significant interest and benefit in the food and pharmaceutical arts as a food product, food ingredient, nutritional supplement or medicament. Such a fiber material would have additional health benefits generally promoting colon health and more specifically for prevention and/or treatment of diseases of the colon. A starch-based composition which combines low glycemic index with controlled-rate of glucose release with the benefits of fermentable fiber would clearly be of significant interest and benefit as a food product, food ingredient, and nutritional supplement for use by individuals for weight control and maintenance, by those with a predisposition to diabetes (prediabetics), for diabetics and more generally by those wishing to generally maintain healthy nutrition and those wishing to maintain or improve their colonic health. Furthermore, such a starch-based composition would be useful as a prophylactic composition to prevent colon disease, or as a pharmaceutical composition or medicament for treatment of diseases of the colon.

### SUMMARY

According to one aspect of the present disclosure, a method of treating constipation is provided. The method includes administering starch-entrapped microbeads.

In yet another aspect of the present disclosure, a method of improving body mass index is provided. The method includes administering starch-entrapped microbeads.

In a further aspect of the present disclosure, a method of improving bowel function is provided. The method includes administering starch-entrapped microbeads.

In another aspect of the present disclosure, a method of improving gut health by increasing butyrate function is provided. The method includes administering starch-entrapped microbeads.

In a further aspect of the present disclosure, a method of promoting prebiotic activity by changing intestinal microbiota is provided. The method includes administering starch-entrapped microbeads.

In yet another aspect of the present disclosure, a method of changing metabolic disease markers in a human is provided. The method includes administering starch-entrapped microbeads to the human.

### BRIEF DESCRIPTION OF DRAWINGS

FIG. 1 shows the relative production of SCFA of starch-entrapped microbeads versus common dietary fibers

FIG. 2 is the fermentation profile of starch-entrapped microbeads (referred to herein as “SM”) versus FOS.

FIG. 3 is the change in bloating over months 1 through 3 of a clinical trial comparing psyllium to different dosages of starch-entrapped microbeads.

FIG. 4 is the change in flatulence over months 1 through 3 of a clinical trial comparing psyllium to different dosages of starch-entrapped microbeads.

FIG. 5 is the change in post-prandial fullness over months 1 through 3 of a clinical trial comparing psyllium to different dosages of starch-entrapped microbeads.

FIG. 6 is the global side effect difference in a clinical trial between psyllium and different dosages of starch-entrapped microbeads.

FIG. 7 shows the microbiota shift comparing starch-entrapped microbeads to resistant starch in a 14 day feeding study using a mouse model.

FIG. 8 is a Biplot of principal coordinates for the microbiome from a clinical trial.

### DETAILED DESCRIPTION

For the purposes of promoting an understanding of the principles of the present disclosure, reference will now be made to the embodiments illustrated in the drawings, and specific language will be used to describe same. It will nevertheless be understood that no limitation of the scope of this disclosure is thereby intended.

The authors of the present disclosure have previously disclosed a starch-based fiber compositions, for example, in US2007/0196437, the contents of which is hereby incorporated by reference in its entirety into the present disclosure.

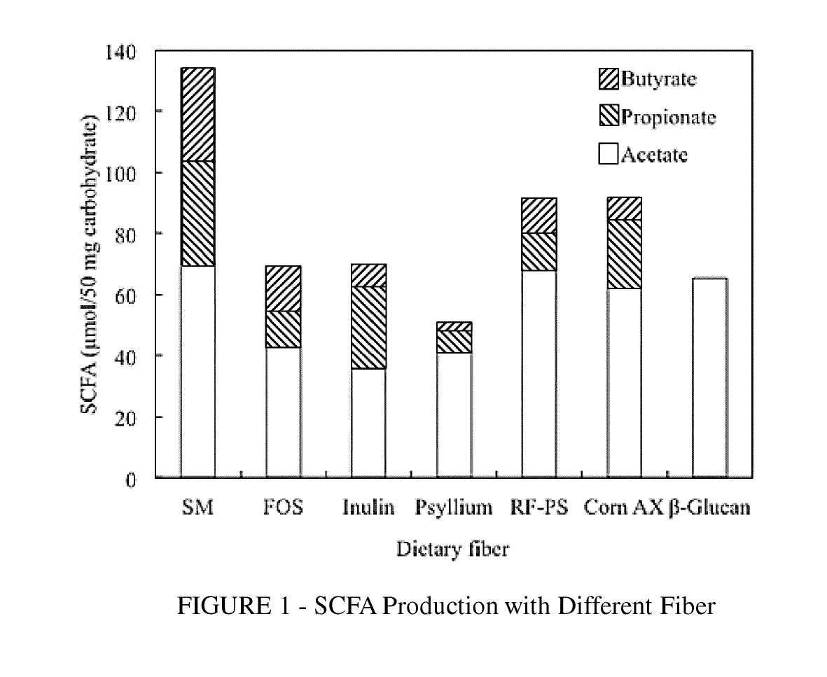

The authors of the present disclosure herein have developed starch-entrapped microbeads (also referred to as a microparticle starch composition) an example of their preparation is set forth in Example 1, whereby starch is placed in a porous gelling non-fermentable polysaccharide matrix which regulates fermentation rate. Administration of starch-entrapped microbeads human fecal microbiota in vitro favors butyrogenic bacteria that results in about a 2-3 fold increase in mol% butyrate (compared to acetate and propionate) compared to other prebiotics in the last half of the fermentation period as shown in FIG. 1 Likewise, when fed to mice, a nearly 3-fold increase in mol% butyrate was observed over resistant starch using fecal samples from the distal colon (FIG. 1). Increased butyrate production is known to correlate with improvements in gut health.

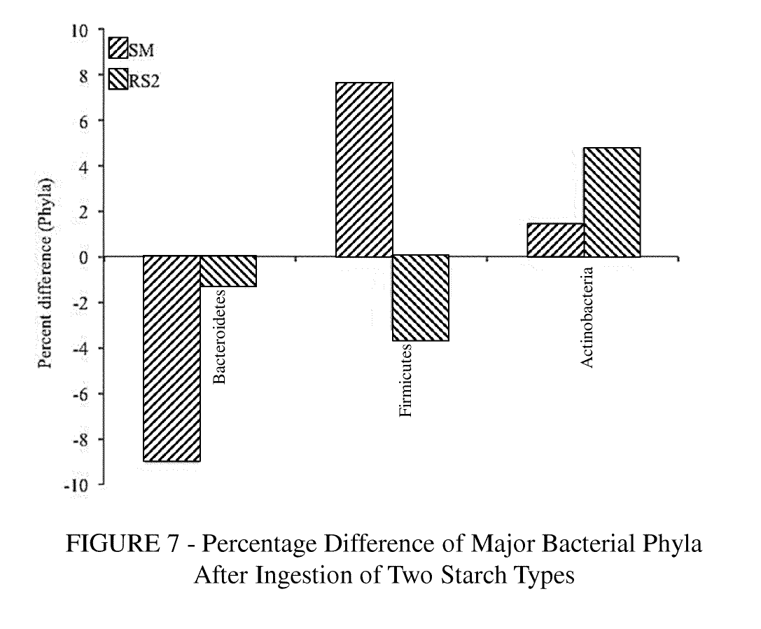

Said starch-entrapped microbeads prebiotics are able to promote gut health by changing the stool microbiota composition favoring saccharolytic bacteria like Bifidobacteria and diminishing proteolytic bacteria like Clostridia in mammals including humans. By changing the intestinal microbiota composition, the authors of the present disclosure show increases in the products of carbohydrate fermentation, such as short chain fatty acids, in particular, butyric acid. Data illustrating such changes in microbiota composition have been shown in the mouse model as set forth in Example 4 and FIG. 7.

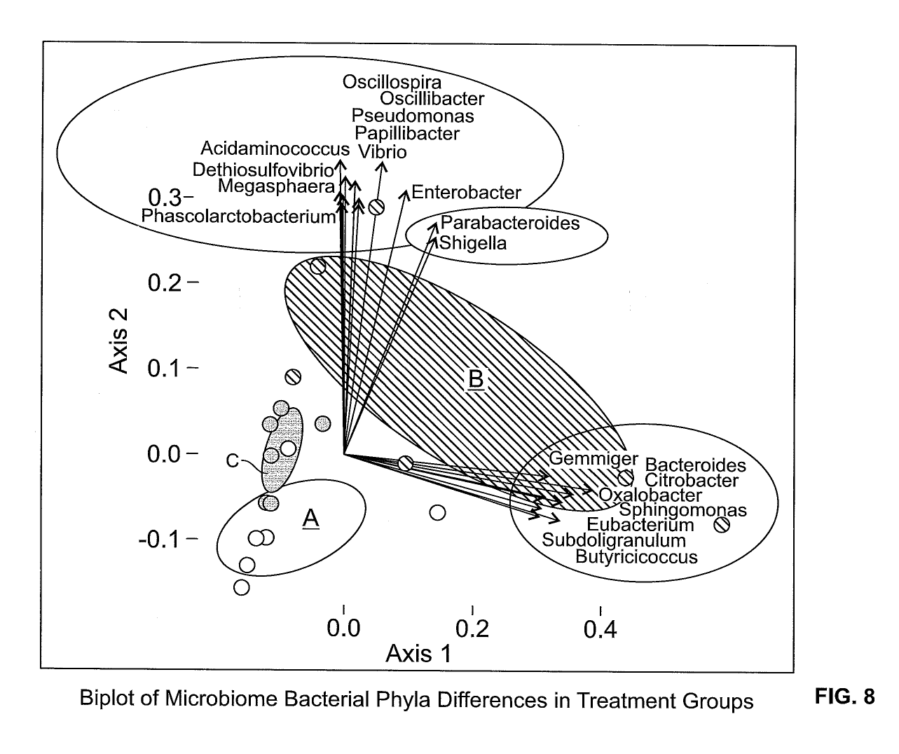

Consumption of starch-entrapped microspheres at both 9 and 12 g differentially modified gut microbiota compared to psyllium. FIG. 8 illustrates that starch-entrapped microspheres impact on the gut microbiota compared to that of psyllium taken from human clinical data.

The starch-entrapped microbeads of the present disclosure can be used to relieve the symptoms of, and treat, for example, constipation. Without being bound by theory, the authors of the present disclosure hypothesize that the improved SCFA production, especially butyrate, contributes to the treatment of constipation. In accordance with the present disclosure, it has been found that starch-entrapped microbeads act to alleviate the symptoms of constipation without fewer side effects than the standard of care as has been shown from human clinical data set forth herein.

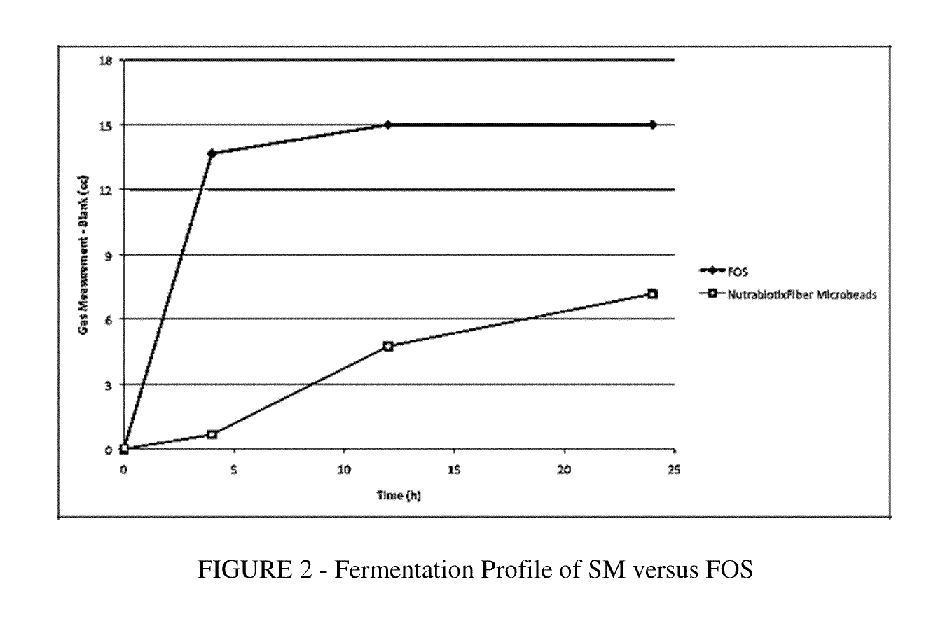

FIG. 2 illustrates the non-bloating properties of starch-entrapped microbeads, in this example, showing a 14 times decrease in gas production at 4 hours as compared to other fibers, such as fructooligosaccharides when these fibers were incubated with human stools.

In one embodiment of the present disclosure, digestive health is improved by administering starch-entrapped microbeads to a mammal in need thereof to treat constipation. In this and other embodiments of the present disclosure, the improvement of and relief of constipation includes treating a mammal in need thereof with about 9 or more grams of starch-entrapped microbeads a day. In another embodiment of the present disclosure, the daily does is between about 9 and about 12 g of starch-entrapped microbeads, including treating with 9 g of fiber a day and 12 grams of Starch-entrapped microbeads a day. Said treatments can be for predetermined periods of time or until symptoms are relieved. In these and other embodiments of the present disclosure constipation may be relieved without bloating compared to psyllium, the standard of care for treating constipation.

The authors of the present disclosure have further disclosed herein that the starch-entrapped microbeads is superior to reducing body mass index when compared with fibers of the prior art.

By “body mass index” what is meant as a measure of body fat based on height and weight which is determined by dividing an individual's body weight by the square of his or her height. In one embodiment of the present disclosure, overall health is improved by administering starch-entrapped microbeads to a mammal in need thereof to reduce body mass index. In this and other embodiments of the present disclosure, the reduction of body fat includes treating a mammal in need thereof with about 9 or more grams of starch-entrapped microbeads a day. In another embodiment of the present disclosure, the daily dose is between about 9 and about 12 g of starch-entrapped microbeads, including treating with 9 g of fiber a day and 12 grams of starch-entrapped microbeads a day. Said treatments can be for predetermined periods of time or until symptoms are relieved.

Example 1 describes a process which may be used for the preparation of starch-entrapped microbeads of the present disclosure. The starch-entrapped microbeads prepared in Example 1 were used in the clinical study experiment described in Example 2.

In Example 2, a three-arm study is described with two arms containing single-agents each consisting of starch-entrapped microbeads of the present disclosure with the third arm consisting of psyllium, a fiber used extensively in the prior art. Example 3 describes results of the clinical trial presented in Example 2.

TABLE 1
Qualitative and Quantitative Measure Response from Patients Taking
Microsphere fiber therapy according to Example 3

| | High | Low | | | |
|---|---|---|---|---|---|
| | Dose | Dose | High | Low | |
| | (n) | (n) | Dose (%) | Dose (%) | Total (%) |
| Positive response | 9 | 12 | 100 | 100 | 100 |
| Negative repsonse | 0 | 0 | 0 | 0 | 0 |
| Significantly better | 8 | 6 | 89 | 50 | 67 |
| response | | | | | |
| Increased TSCFA | 5 | 7 | 50 | 58 | 55 |
| Increased mole % BA | 4 | 7 | 40 | 58 | 50 |
| Increased absolute BA | 2 | 6 | 20 | 50 | 36 |

Table 1 provides data arising out of the clinical trial data from Example 3. According to these data, all clinical trial subjects dosed with the starch-entrapped microbeads of the present disclosure reported improvement in constipation symptoms with not a single patient reporting a worsening of symptoms despite not controlling diet. Sixty-seven percent of the population reported significantly better bowel function, consistency, ease or overall well-being with the high-dose cohort reporting a higher (89% versus 50%) symptom response as compared to the low-dose cohort.

Table 1 also shows an increase in total short chain fatty acid, mole percent butyric acid and absolute butyric acid at both high and low doses, 55%, 58%, and 50% in the total, low and high doses, respectively when compared with pre-treatment analyses. Mole percent butyric acid also increased in 50% of the population, while absolute butyric acid increased in over one-third of the group.

Preliminary data also show superiority to psyllium in absolute butyric acid production and body fat percentage improvement. When starch-entrapped microbeads of the present disclosure were compared to psyllium, in particular, absolute BA increased in only 14% of the psyllium dosed population while the starch-entrapped microbeads group exhibited an increase in 36% overall with 20% being in the high dose cohort and 50% in the low dose cohort. These data further show an overall loss of BMI from the after taking the starch-entrapped microbeads with an average loss of 2.8% occurring in 64% of that population whereas am average BMI increase in those taking psyllium was 4.4% occurring in 80% of the psyllium population.

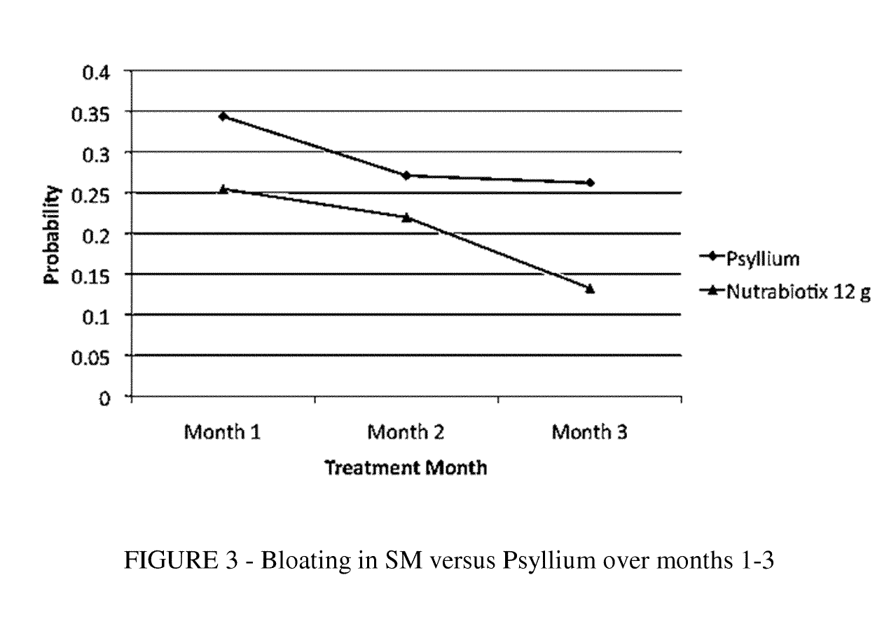

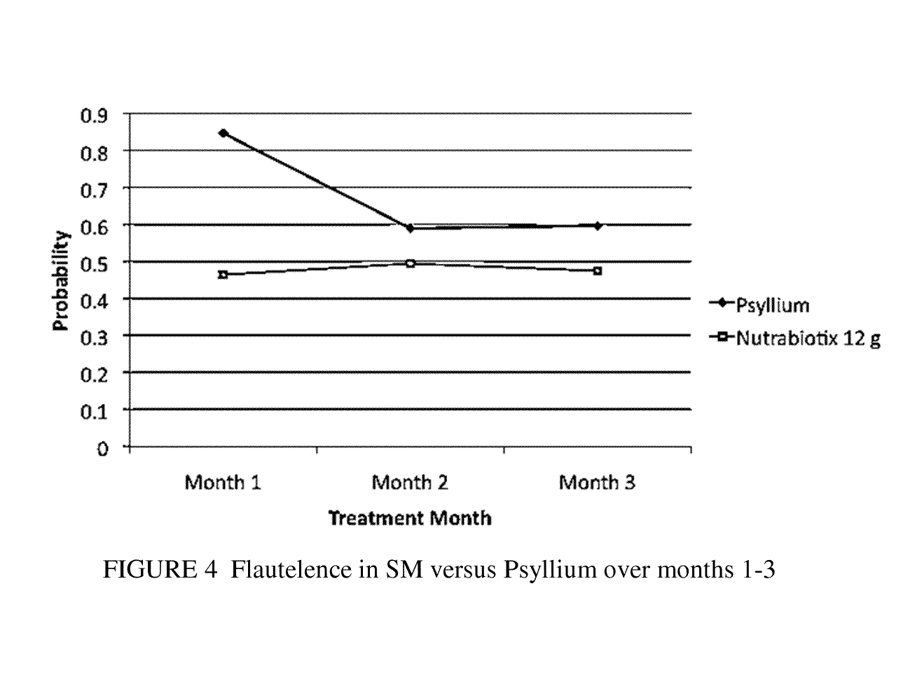

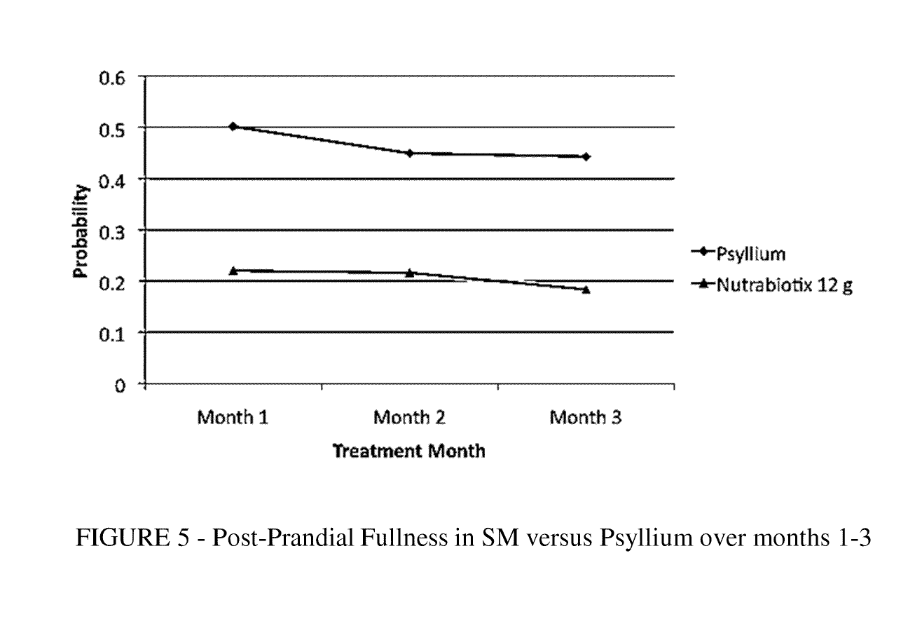

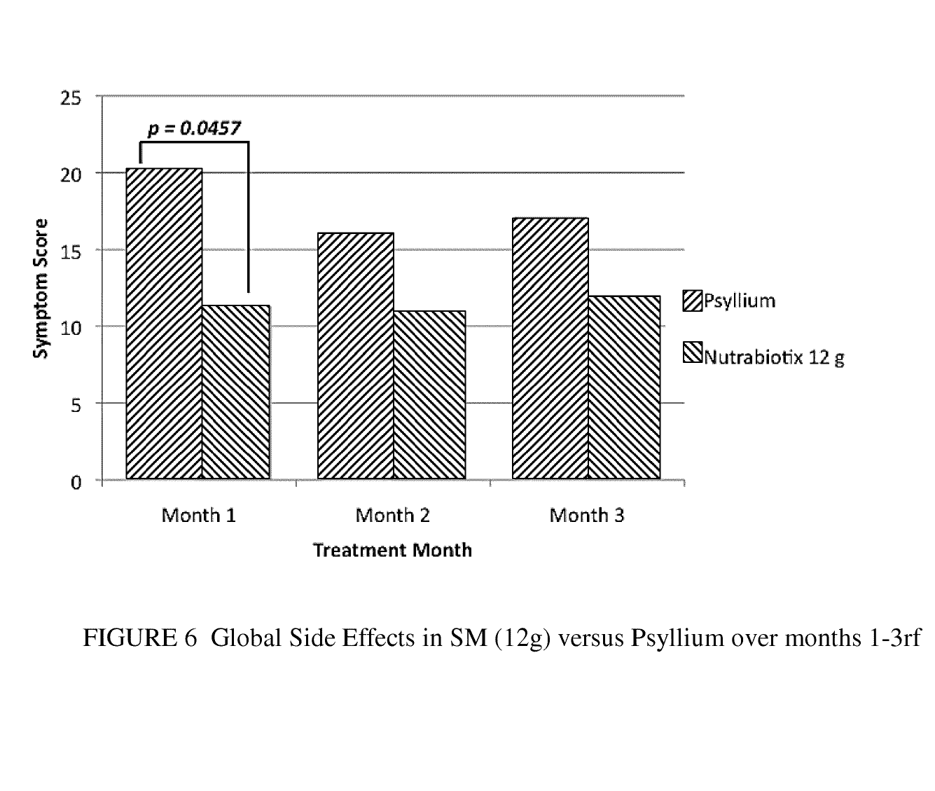

The clinical results further reveal that at the dose of 12 g per day of starch entrapped microbeads, a marked increase in SFCA production by gut microbiota was observed and positively impacted microbiota composition, fewer side effects were reported by patients taking the starch entrapped microbeads than psyllium. For example, FIG. 3 shows continued decrease in reported bloating by patients taking the 12 g dose of the starch entrapped microbeads versus psyllium. In FIG. 4, a lower probability of flatulence in this patient group was also observed and in FIG. 5, reports of post-prandial fullness was similarly reported. In sum, these data show, in FIG. 6, that the 12 g dose of the starch-entrapped microbeads exhibited a statically significant decrease in side effects over psyllium. A dose at 9 g was also provided to patients, but the data were insufficient to show any statistically meaningful difference with respect to psyllium.

As noted, the starch-entrapped microbeads change microbiota in the gut. FIG. 7 shows that when comparing raw potato starch to the starch entrapped microspheres in Example 4 it was found that the starch-entrapped microspheres enhanced the production of Firmicutes and lowered the production of Bacteroidetes. Other bacteria that have shown to be enhanced are Firmicutes phylum, Clostridia and Erysipelotrichi classes, Clostridiales and Erysipelotrichales orders, Clostridiaceae and Erysipelotrichaceae families, Clostridium and Turicibacter geni, and Clostridium and Turicibacter species.

The starch-entrapped microbeads also showed additional clinical efficacy. Dosing with the starch entrapped microbeads was found to reduce insulin levels and non-HDL cholesterol in clinical subjects as set forth in Example 5.

The starch-entrapped microbeads also showed additional clinical efficacy that extend beyond that of gut health. Dosing with the starch entrapped microbeads for 3 months reduced both low-density lipoprotein (LDL) cholesterol and non-high-density lipoprotein cholesterol (non-HDL) at both the 9 and 12 g doses when a subset of subjects with abnormal serum values were examined. A summary of the data are set forth in table 2 below.

TABLE 2
Abnormal Serum Values (% Change)

| | Triglycerides | HDL | LDL | | Insulin |
|---|---|---|---|---|---|
| | mg/dL | mg/dL | mg/dL | non-HDL | uU/ml |
| Psyllium | 9.52 | 6.56 | −12.52 | −17.43 | −7.77 |
| 9 g NTX | −7.76 | 1.73 | 1.53 | −7.10 | 1.84 |
| 12 g NTX | −1.05 | 4.60 | −13.50 | −13.05 | 3.74 |

An increase in HDL cholesterol and reduction of serum triglycerides were seen, indicating that supplementation with starch-entrapped microbeads may serve as an alternate lipid-modifying dietary compound that can have a beneficial impact on systemic health.

### EXAMPLES

### Example 1

### Preparation of Starch-Fiber Composition Used in Examples 2 and 3

All material was food grade. A starch and alginate solution [1% (w/v) alginate and 10% (w/v) starch] was prepared by mixing heated deionized water (100 deg F.) with sodium alginate and corn starch. The sodium alginate was mixed first until complete dissolution occurred. The starch was added to the alginate mixture until the starch was completely mixed. A 2% (w/v) calcium chloride solution was mixed and set aside. The starch/alginate solution was pumped using precision metering pumps to provide constant flow and microparticle drop formation through a precision nozzle assembly. The microparticles were allowed to drop into the calcium chloride solution and allowed to gel. The gelled microparticles were washed thoroughly with deionized water. These microparticles were then dried in a 45 deg C. oven for at least 24 hours. The dried microparticles were sieved to yield less than 1000 micrometer sized microparticles. The microparticles were capsuled in gelatin capsules and packaged in sealed containers for human use.

### Example 2

### Clinical Protocol for Delivery of Starch-Fiber Compositions

A double-blind clinical study design was implemented to investigate the fiber health properties of a starch-fiber composition. To date, thirty-three otherwise healthy, asymptomatic subjects with body mass indices of 20-30 having self-described unsatisfactory bowel habits and suffering from constipation were divided into three arms. In arm I, subjects were given 4 capsules containing 1 gram psyllium fiber 3 times a day (12 g total per day). In arm II, subjects were given 3 capsules of 1 g each of Starch-entrapped microbeads prepared according to Example 1, three times daily for a total of 9 g per day. In arm 3, subjects were given 4 capsules of 1 g each of Starch-entrapped microbeads prepared according to Example 1, three times daily for a total of 12 g per day. Twenty-two subjects to date have been dosed in arms 2 and 3. Patients were on each arm for a period of 18 weeks. Clinical endpoints of the study were designed to be body mass index improvement, side effect tolerance, prebiotic activity in the form of short chain fatty acid in the stool, bacterial interrogation of the stool, and stool weight.

### Example 3

### Clinical Results from Example 2

Table 1 is an interim patient summary data comparing arms II and III of the clinical trial after the dosing regimen with patient data collected prior to the dosing regimen. The High Dose column refers to arm III of the clinical trial of Example 2 wherein trial subjects received 12 grams of a starch-entrapped microbeads daily and the Low Dose column refers to arm II which received 9 grams daily. TSCFA refers to total short chain fatty acid and BA refers to butyric acid.

Qualitative responses were measured by clinical trial subject diary entries at various time points during the dosing. Patients were asked to self-report if their symptoms (bowel function, consistency, ease and overall wellbeing) were significantly worse, minimally worse, minimally better or significantly better. The “positive” response value in table 1 sums the minimally better and significantly better responses. The “negative” response value in table 1 sums the significantly worse and minimally worse responses.

Quantitative measurements were obtained through fecal fermentation analysis of short chain fatty acids as described by Rose, et al. in J. Agric. Food Chem. 2010, 58, 493-499. Fecal samples from clinical trial subjects were obtained.

Body Mass Index measurements were taken at the second visit for each trial subject, which occurred immediately before dosing began and then again at visit 3 which occurred after the regimen of the clinical trial had been completed. The methodology for measuring BMI was Obtaining the patients weight in pounds using a medical scale and also measuring the patients height in feet and inches; these parameters were used to calculate BMI using the formula BMI=[weight(lb)*4.88]/[height(ft)]̂2.

Ongoing clinical trial primary outcome measures also include better prebiotic activity and the production of more bifidobacteria and/or other beneficial bacteria. Increasing bifidobacteria may result in decreasing proteolytic bacteria such as clostridia.

### Example 4

Twenty male mice aged 6 weeks with an average weight of 21.80±0.35 g were acquired and housed in individual cages containing wood pulp bedding. The cages were kept in an isolated room at a temperature of 22-25° C. with a 12 h light/dark cycle. Animals were acclimatized for a period of one week during which they had free access to water and semisynthetic feed as per the guidelines of the American Institute of Nutrition (AIN). Test diets were manufactured commercially into ½ inch pellets. Changes in composition based on the AIN 76A diet were 5% cellulose and 15% corn starch were replaced by 10% (w/w) dietary fiber of interest and 10% sucrose. Sucrose was used as a replacement because it does not reach the large intestine for fermentation and was the major single component of the acclimatizing mouse chow. Before incorporating these substrates into animal diets, total carbohydrate was determined using the phenol-sulfuric acid method. Because alginate is not fermented by gut microbiota, total starch instead of total carbohydrate was determined in starch-entrapped microspheres using a commercial kit according to the manufacturer's instructions. Based on the amount of total carbohydrate, 10% equivalent carbohydrate was incorporated into diet. The two substrates tested in this study included starch-entrapped microbeads (“SM”) and raw potato starch (RS2). Animals were randomly assigned to two diet groups. Mice were raised on these diets for two weeks. During the 2 week period, animals were monitored daily for changes in fecal consistency and general wellbeing. Body weight was measured every third day as an indicator of feed intake and health. Fecal samples were collected before the start of the feeding trial (day 0) and were continued to collect at day 3, 7, 11, and 14 (last day of the study). At the end of day 14, animals were euthanized. Fecal DNA material was isolated and analyzed using pyrosequencing microbial interrogation analysis, the results of which are shown in FIG. 7. This data show differences between final (day 14) and initial (day 0) percentages in major bacterial phyla, classes, orders, families, genera, and species. Phylum level changes were observed only in the most abundant phyla namely, Bacteroidetes, Firmicutes, and Actinobacteria. Starch-entrapped microspheres promoted Firmicutes and greatly reduced Bacteroidetes populations, whereas RS2 led to a decrease in Firmicutes and had little effect on the Bacteroidetes population.

### Example 5

In a randomized, double-blind, placebo controlled, ongoing phase I clinical trial, we evaluated the effect of a prebiotic (starch-entrapped microbeads) on bowel habits in healthy individuals. We have analyzed microbiome composition with 16s rDNA sequencing and microbiome differences were compared with weighted Unifrac distance base RDA (wU-dbRDA). A biplot of the wU-db RDA ordination and the most common genera is shown in FIG. 8 (arrows indicate the contribution of individual taxa to the dbRDA axes). A, B and C denote the study groups that are receiving psyllium (A), microbeads at high dose (12 g) (B), and microbeads at low dose (9 g) (C). Data show that these starch-entrapped microbeads increase fecal SCFA concentrations. Evidence for impact on metabolic disease markers were also seen; prebiotic treatment reduced insulin levels in 5 of the 8 subjects that had elevated insulin at baseline. In addition, when provided 12 g starch-entrapped microbeads, subjects in the highest tertile had an average reduction in non-HDL cholesterol by 43%.

Those skilled in the art will recognize that numerous modifications can be made to the specific implementations described above. To implementations should not be limited to the particular limitations described. Other limitations may be possible. Therefore, the following claims are not to be limited to the specific embodiments illustrated and described above. The claims, as originally presented and as they may be amended, encompass variations, alternatives, modifications, improvements, equivalents, and substantial equivalents of the embodiments and teachings disclosed herein, including those that are presently unforeseen or unappreciated, and that, for example, may arise from applicants/patentees and others.

1. A method of treating constipation for a human or animal in need thereof comprising administering starch-entrapped microbeads.

2. The method of claim 1 wherein between about 9 and about 12 g of starch-entrapped microbeads are administered daily.

3. The method of claim 1 or 2 wherein the treatment occurs with less bloating to the human or animal when compared to psyllium.

4. A method of altering microbiotic composition of a mammal by administering starch-entrapped microbeads to the mammal.

5. The method of claim 4 wherein the mammal is human.

6. The method of claim 1 or 4 wherein the number of short-chain fatty acid producing bacteria is enhanced.

7. The method of claim 6 wherein the bacteria enhanced are Firmicutes phylum, Clostridia and Erysipelotrichi classes, Clostridiales and Erysipelotrichales orders, Clostridiaceae and Erysipelotrichaceae families, Clostridium and Turicibacter geni, and Clostridium and Turicibacter species.

8. A method of changing metabolic disease markers in a human by administering starch-entrapped microbeads to the human.

9. The method of claim 8 wherein the metabolic disease markers are insulin or HDL cholesterol.

10. The method of claim 9 wherein the insulin or HDL cholesterol markers or both are reduced.

11. The method of claim 10 wherein between about 9 and about 12 g of starch-entrapped microbeads is administered daily.

12. The method of claim 1 or 8 wherein the amount of starch-entrapped microbeads is greater than 12 g administered daily.
